(19) United States
(12) Patent Application Publication
(10) Pub. No.: US 2009/0299357 A1
(43) Pub. Date: Dec. 3, 2009

(54) CRYONEEDLE AND CRYOTHERAPHY SYSTEM

(75) Inventor: Linqiu Zhou, Wynnewood, PA (US)

Correspondence Address: NIXON PEABODY, LLP, 401 9TH STREET, NW, SUITE 900, WASHINGTON, DC, 20004-2128, US

(73) Assignee: THOMAS JEFFERSON UNIVERSITY, Philadelphia, PA (US)

(21) Appl. No.: 12/298,261

(22) PCT Filed: Apr. 2, 2007
(86) PCT No.: PCT/US07/08177

§ 371 Date: Jun. 11, 2009

Related U.S. Application Data
(60) Provisional application No. 60/794,107, filed on Apr. 24, 2006.

(51) Int. Cl. A61B 18/02

(52) U.S. Cl. 606/21

(57) ABSTRACT

A device and system for cryogenically treating tissue having an external cryoneedle including an outer housing having a proximal end, a proximal portion, a distal portion and a distal end. The proximal portion of the outer housing is coated with a first material and the distal portion of the outer housing is coated with a second material. The distal end is sealed with a third material. The first material has different temperature conductive properties than the second and third materials. An internal cryoneedle having a first and second end is axially disposed within the external cryoneedle. The first end of the internal cryoneedle is in communication with the sealed distal end of the external cryoneedle and the second end of the internal cryoneedle being in fluid communication with a cryogen source.

### BACKGROUND OF THE INVENTION

1. Field of the Invention

The present invention relates to deep cold cryoneedle and system for percutaneous cryotherapy in pain management, and more particularly to a cryotherapy system including the cryoneedle, cryotherapy machine and controller.

2. Description of the Related Art

Cold for analgesia has been used for thousands of years. Modern cryoanalgesia debuted in the early 1960's. Recent studies about cryoanalgesia have shown that different temperatures of the cryolesion result in different degrees of nerve damage. Colder temperatures correlate with longer recovery times, but achieve a longer duration of pain relief.

Current treatments using cryoanalgesia include spinal dorsal ramus (lower back pain), cervical spinal dorsal ramus (neck pain), facial neuralgia (facial nerve), trigeminal neuralgia, intercostals nerve (chest wall pain), neuroma, and ilioinguinal, iliohypogastric and genitofemoral subgastric neuralgia. Cryolesion applications include malignant tumor therapy for prostate, liver and skin cancers, as well as renal tumors.

Cryoanalgesia is a safe, less painful procedure with no complications of neuroma, neurolitis, parathesia and chemical toxicity in comparison with radiofrequency neurolysis and chemical neurolysis (phenol or alcohol).

Cryoblation of peripheral nerves is less expensive and provides longer spasm free periods with no botulinum toxin toxicity.

Moreover, cryolesion is a reversible process. The nerve will regenerate after cryoanalgesia, because the cryolesion does not damage the basal membrane of the nerve.

FIGS. 1A-1C illustrate a known cryoprobe used for the percutaneous treatment of cancer. The needle is a MR imaging-compatible 2.2 mm diameter (8 gauge) needle, approximately 16 cm in length. The needle is specially designed for MR imaging-guided percutaneous cryotherapy. FIG. 1B is a close-up view of the cryoneedle tip. As shown in FIG. 1C, an ice ball is formed on the tip after a 15 minute freeze time in 8 ounces of water. The ice ball had a lateral diameter of approximately 3.25 cm. Due to the large size of this cryoprobe, an open procedure is required to deliver cryotherapy. The uncoated needle body (cold needle body) can also cause standby tissue damage.

There are limitations with the known cryoanalgesia devices. The large sizes of current cryoprobes, i.e., ranging from 10 to 15 gauge needles (1.4 to 2 mm), technically limit clinical practice. See the cryosurgical probe and sheath disclosed in U.S. Pat. No. 6,475,212.

Moreover, the operating temperatures of these current devices are not cold enough to produce the desired complete and longer pain relief. For example, currently available operating temperatures only range between −20 to 89° C. U.S. Pat. No. 6,936,048 discloses a cryoblation needle having a gauge of 16 to 18. However, the device is not operable at a deep cold temperature.

Another disadvantage of the prior art probes is that the temperature of the tip is non-adjustable. This constant temperature limits the ability to coordinate the device with different clinical purposes, such as the treatment of pain or of a tumor.

There are not only limitations with the current cryoprobe devices themselves. The coordinating systems or machines cannot generate deep cold temperatures. For example, the SL2000 Lloyd Neurostat cryoprobe manufactured by Westco Medical Corp., produces a maximum −20° C. Moreover, due to the typical large size of the systems they are often importable.

Thus, there is a need for a cryoneedle and system that can operate at deep cold temperatures and not damage tissues not targeted for the cryoanalgesia.

### SUMMARY OF THE INVENTION

One aspect of the present invention is to provide a cryotherapy device and system for percutaneous cryotherapy of pain management.

Another aspect of the present invention is to provide a deep-cold cryotherapy device and system for percutaneous peripheral nerve cryoblation that can be used for the treatment of spasticity, such as SCI, TBI and CP induced spasm.

Yet another aspect of the present invention is to provide a cryotherapy device and system for cryotherapy treatment of both malignant and benign tumors.

One advantage of the deep-cold cryotherapy device of the present invention is the small size of the needle.

Another advantage of the cryotherapy device of the present invention is that the temperature of the needle tip operates at temperatures below −100° C., much cooler than temperatures of current cryoprobes. The cryotherapy temperature of the tip is also automatically controlled.

By configuring the cryoprobe with different materials, the tip of the needle can be much colder than the rest of the probe. Moreover, a protective coating of Teflon® on a proximal portion of the needle prevents standby tissue cryolesion.

The supportive cryotherapy machine is smaller than current N₂O cryotherapy machines. The resulting smaller weight and size makes the machine more portable and convenient to use.

The flow rate of the high-pressure gas of the cryotherapy system of the present invention can be controlled by presetting the temperature.

In accomplishing these and other aspects of the present invention there is provided a device for cryogenically treating tissue having an external cryoneedle including an outer housing having a proximal end, a proximal portion, a distal portion and a distal end. The proximal portion of the outer housing is coated with a first material and the distal portion of the outer housing is coated with a second material. The distal end is sealed with a third material. The first material has different temperature conductive properties than the second and third materials. An internal cryoneedle having a first and second end is axially disposed within the external cryoneedle. The first end of the internal cryoneedle is in communication with the sealed distal end of the external cryoneedle and the second end of the internal cryoneedle being in fluid communication with a cryogen source.

In accomplishing these and other aspects of the present invention there is also provided a cryotherapy system for treating tissue having an external cryoneedle including an outer housing having a proximal end and a distal end. The outer housing is coated with a first material in approximation to the proximal end and a second material in approximation to the distal end. The distal end is sealed with a third material, wherein the first material has a different temperature conductive property than the second and third materials. An internal cryoneedle having a first and second end is axially disposed within the external cryoneedle. The first end of the internal cryoneedle is in communication with the sealed distal end of the external cryoneedle. A pressurized source of cryogen is provided and the second end of the internal cryoneedle is in fluid communication with the cryogen source. A temperature and feedback control device is in communication with the cryogen source and the external cryoneedle.

In accomplishing these and other aspects of the present invention there is also provided a deep cold cryotherapy method for treating a tissue including the step of percutaneously inserting a cryotherapy device into a patient. The cryotherapy device includes an external cryoneedle having an outer housing with a proximal end and a distal end. The outer housing is coated with a first material in approximation to the proximal end and a second material in approximation to the distal end. The distal end is sealed with a third material. The first material has different temperature conductive properties than the second and third materials. An internal cryoneedle is axially disposed within the external cryoneedle. The first end of the internal cryoneedle is in communication with the sealed distal end of the external cryoneedle. A cryogen is delivered to the first end of the internal needle, and the third material at the distal end of the external needle is cooled to −100° C. or less to freeze the tissue.

These and other features, aspects, and advantages of the present invention will become more apparent from the following detailed description of the preferred embodiment relative to the accompanied drawings, in which:

### BRIEF DESCRIPTION OF THE DRAWINGS

FIGS. 1A-1C are photographs of a known cryoprobe for cancer therapy.

FIG. 2 is a side view of the internal needle of the cryoprobe device of the present invention.

FIG. 3 is a cross-sectional view of the external needle of the cryoprobe device of the present invention.

FIG. 4 is an enlarged cross-sectional view of the operating end of the cryoprobe device of the present invention.

FIG. 5 is a schematic view of the deep-cold cryotherapy system of the present invention.

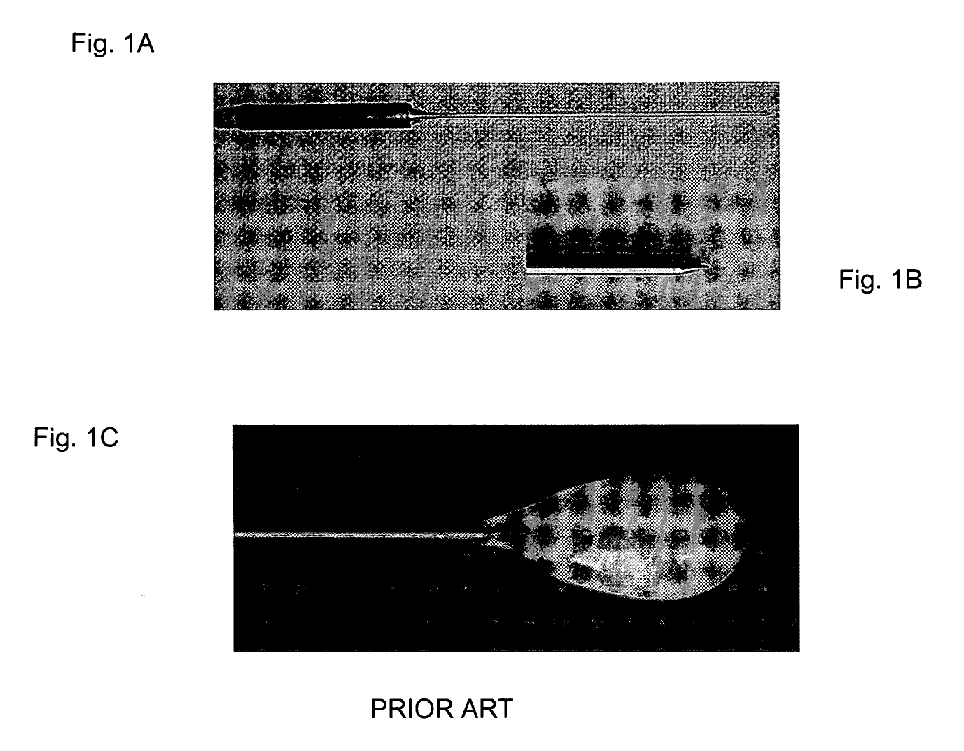

### DETAILED DESCRIPTION OF THE PREFERRED EMBODIMENTS

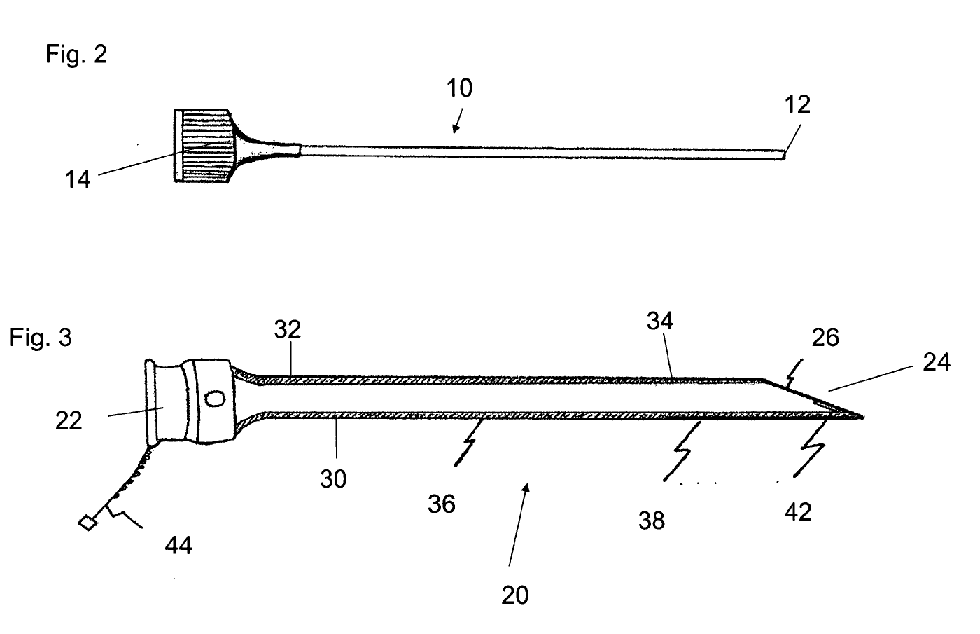

Referring to FIGS. 2 and 3, the cryoprobe device of the present invention includes two needles, an internal needle 10 for delivering the cryogen gas and an external needle 20. External needle 20 can be a modified 18 gauge spinal needle and has a proximal end 22 and a distal end 24. Distal end 24 has a sharpened tip 26. External needle 20 has an outer housing 30 having a proximal body portion 32 and a distal portion 34. The proximal portion 32, for example, a 60 mm length of the needle is coated with a first thermally non-conductive material 36 such as Teflon® or other plastic or insulating material. The coating 36 protects the tissue surrounding this portion of the needle from cryolesion. The coating can be polished to a thickness of about 20-30 μm. It should be appreciated that different thicknesses, materials and coverage of the needle are incorporated by the present invention.

External needle 20 has an outer diameter of 1.3 mm (18 gauge) or range of gauges from 15-18. A needle having such a small size enables the physician to perform cryotherapy percutaneously. It should be appreciated that the present invention can also be used in non-percutaneous procedures.

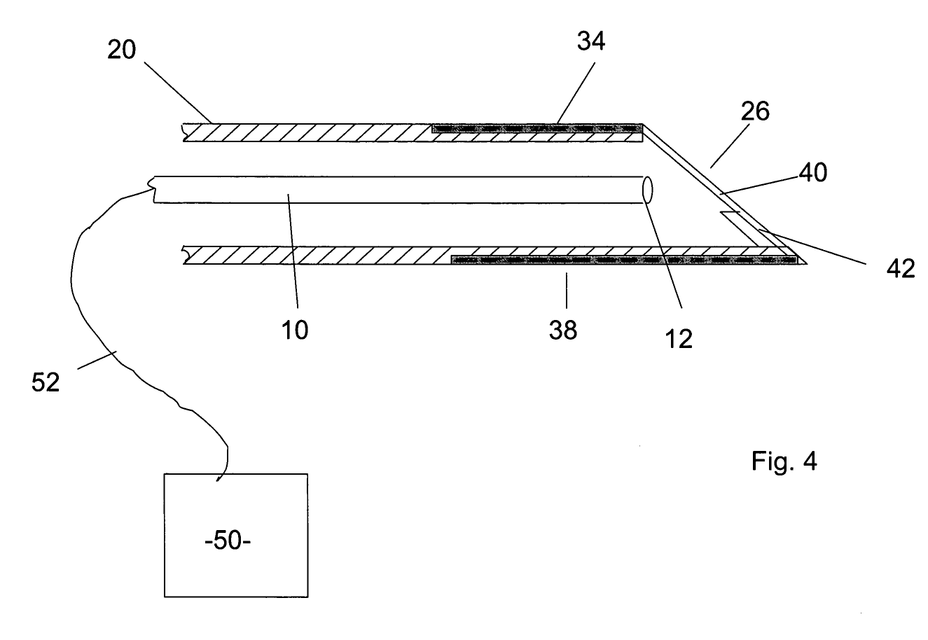

As is shown in FIG. 4, the distal portion 34 of external needle 20 includes a coating of a second material 38. Material 38 is a thermally conductive material, such as a metal, preferably silver, electroplated, coated or otherwise formed on the distal portion in approximation to the tip of the needle. It should be appreciated that different types of conductive materials are contemplated. Tip 26 is sealed with a thermally conductive material 40. Material 40 can be a metal, preferably silver. The tip is welded with the silver approximately about 80-100 μm. Because coating 36 acts as an insulator and materials 38 and 40 act as thermal conductors, the different thermal conductivity properties of the material allow the device to function at deep cold temperatures of −100° C. or less. The temperature control system automatically facilitates the tip of needle reaching to the presetting temperature (from −20° C. to −180° C.).

Located at the tip 26 is a temperature sensor or thermocouple 42. as will be described further herein, the system of the present invention can automatically control the cryotherapy temperature by detecting the temperature of the needle tip. If the temperature is high the system will increase the pressure and flow rate of the cryogen until the temperature of the tip reaches the preset temperature.

Referring again to FIG. 3, the temperature sensor 42 includes sensor wires 44 connected to the sensor 42 that extend from a hub 46 at the proximal end 22 of external needle 20. As will be described further herein, the sensor wires communicate with a temperature measurement unit in the cryotherapy system for identifying the temperature sensed by the sensor 42.

Referring back to FIG. 2, internal needle 10 has a first end 12 and a second end 14. As shown in FIG. 4, internal cryoneedle 10 is axially disposed within external needle 20. Internal needle 10 can be a modified 25 gauge spinal needle and is used to conduct the pressurized cryogen gas. Internal needle 10 is shorter than external needle 20. First end 12 of the internal cryoneedle is in communication with the sealed tip 26 of the external cryoneedle. The second end 14 of the internal cryoneedle is connected to a cryogen source 54 by a supply hose 52. Hose 52 can also be coated with Teflon® to limit heat transfer.

Internal needle 10 acts as a cryogen inlet tube to deliver the cryogen to the tip 26. The gas then travels back to the outlet vent through space between the internal and external needles. The outlet vent is located at the proximal end of the external needle to allow the cryogen gas to exit to air.

The cryogen can be a high-pressure N₂O gas or other equivalent fluid that can reach the desired cold temperatures. As will be described further herein, the high-pressure N₂O gas is pumped through hose 52 and conducted to inner needle 10. At the tip of the inner needle the N₂O gas is rapidly expanded due to the Joule-Thompson Effect, which extracts heat from the high heat conducting needle tip and generates a deep-cold temperature of −100 to −180° C. As described above, the temperature of the needle tip is adjustable depending on the flow rate of the gas.

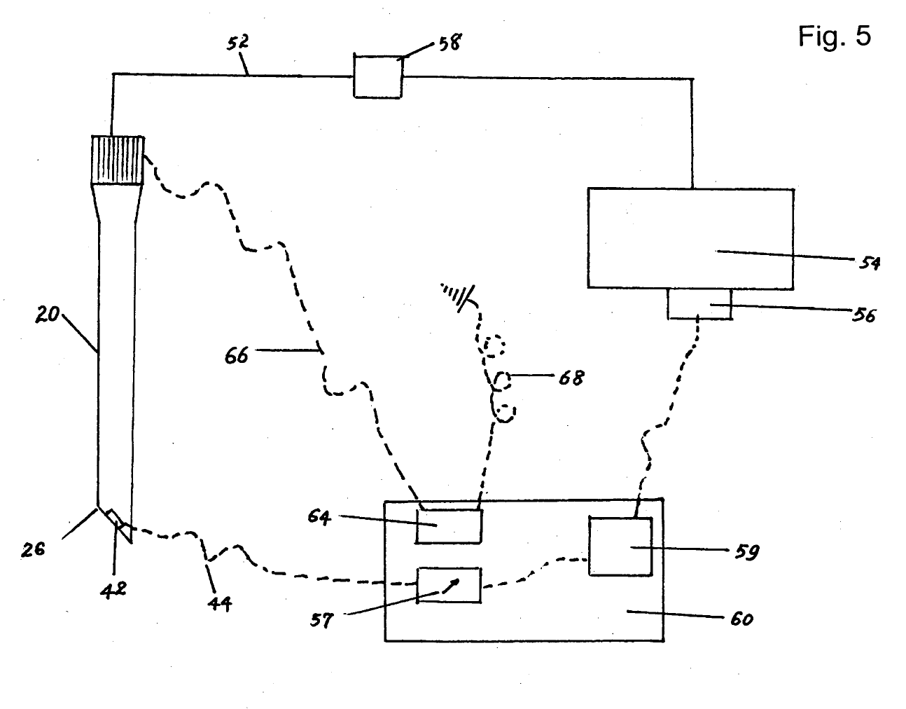

FIG. 5 illustrates the cryotherapy system of the present invention. Cryogen source 54 is preferably a pressurized container connected to hose 52 that also includes a handle control device 58. The handle control device 58 can be a valve system that controls the cryogen flow and adjusts the needle tip temperature. Cryogen source 54 can be a 5-gallon container of liquified nitrogen. This enables the cryotherapy machine to be smaller and more portable. The cryogen container includes a pumping mechanism 56, which adjusts the cryogen pressure based on the presetting temperature and feedback temperature from the needle tip, such pressure can reach to 600-800 psi.

Additionally, as described herein the machine of the present invention includes a temperature monitoring system or a temperature and feedback control device in communication with the cryogen source and the external cryoneedle. The temperature and feedback control device includes sensor 42, sensor wires 44 and a temperature monitor 59 that is part of a microprocessor or computer system 60. The system can also include a temperature gauge 57. Monitor 59 detects the temperature of sensor or probe 42 and feeds back to the automatic pressure adjustable device 56 to modify the pressure of the cryogen based on the preset needle tip temperature.

The system also includes a nerve stimulator component that includes a stimulating generator 64, a stimulator electrode 26 and a ground electrode 68. The stimulating generator 64 contains electrical stimulator, which generates different electrical currents to stimulate sensory or motor nerves, and impedance detector which checks tissue conduction and blood flow. The cryoneedle coated with Teflon and connected with wire 66 to the stimulator is used as cathode to localize nerve. A ground electrode (pad, 68) with wire connecting to the stimulator is attached to skin.

In use, the cryoprobe is inserted percutaneously into the patient. The sharpened tip 26 enables the tip to be inserted without invasive incisions. The cryogen is delivered to the tissue site to freeze the nerve, tumor or other tissue. Depending on the preset temperature of the tip, the system will automatically adjust the flow rate of the cryogen to adjust the temperature accordingly. The different conductive properties of the materials of the external needle of the probe simultaneously protect the surrounding tissue and apply a deep-cold treatment to the tissue at the tip.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A device for cryogenically treating tissue comprising:
an external cryoneedle including an outer housing having a proximal end, a proximal portion, a distal portion and a distal end, the proximal portion of the outer housing being coated with a first material and the distal portion of the outer housing being coated with a second material, and wherein the distal end is sealed with a third material, the first material having different temperature conductive properties than the second and third materials; and
an internal cryoneedle having a first and second end axially disposed within the external cryoneedle, the first end of the internal cryoneedle being in communication with the sealed distal end of the external cryoneedle and the second end of the internal cryoneedle being in fluid communication with a cryogen source.

2. The device of claim 1, wherein the external needle has an outer diameter 1.3 mm.

3. The device of claim 1, wherein the operating temperature of the device is −20° C. to −180° C.

4. The device of claim 1, wherein the first material is Teflon®.

5. The device of claim 4, wherein the second material is a layer of silver electroplated on said distal portion.

6. The device of claim 5, wherein the third material is silver.

7. The device of claim 5, wherein the electroplated silver layer has a thickness of approximately 20-30 μm.

8. The device of claim 1, further comprising a thermocouple located at the distal end of the external cryoneedle.

9. The device of claim 8, further comprising a nerve stimulator located at the distal end of the external cryoneedle.

10. A cryotherapy system for treating tissue comprising:
an external cryoneedle including an outer housing having a proximal end and a distal end, the outer housing being coated with a first material in approximation to the proximal end and a second material in approximation to the distal end, and wherein the distal end is sealed with a third material, the first material having different temperature conductive properties than the second and third materials;
an internal cryoneedle having a first and second end axially disposed within the external cryoneedle, the first end of the internal cryoneedle being in communication with the sealed distal end of the external cryoneedle;
a pressurized source of cryogen, wherein the second end of the internal cryoneedle is in fluid communication with the cryogen source; and
a temperature and feedback control device in communication with the cryogen source and the external cryoneedle.

11. The device of claim 10, wherein the external needle has an outer diameter 1.3 mm.

12. The device of claim 10, wherein the operating temperature of the device is −20° C. to 180° C.

13. The device of claim 10, wherein the first material is Teflon®.

14. The device of claim 13, wherein the second material is a layer of silver electroplated on said distal portion.

15. The device of claim 14, wherein the third material is silver.

16. The device of claim 15, wherein the electroplated silver layer has a thickness of approximately 20-30 μm.

17. The device of claim 10, further comprising a thermocouple located at the distal end of the external cryoneedle, and wherein the temperature and feedback control device is in communication with the cryogen source and the thermocouple.

18. The device of claim 10, further comprising a nerve stimulator located at the distal end of the external cryoneedle.

19. The device of claim 18, wherein the temperature and feedback control device is part of a microprocessor in communication with the nerve stimulator and a thermocouple located at the distal end of the external cryoneedle.

20. A deep cold cryotherapy method for treating a tissue comprising the steps of:
inserting a cryotherapy device into a patient; the cryotherapy device including an external cryoneedle including an outer housing having a proximal end and a distal end, the outer housing being coated with a first material in approximation to the proximal end and a second material in approximation to the distal end, and wherein the distal end is sealed with a third material, the first material having different temperature conductive properties than the second and third materials, and an internal cryoneedle having a first and second end axially disposed within the external cryoneedle, the first end of the internal cryoneedle being in communication with the sealed distal end of the external cryoneedle;
delivering a cryogen to the first end of the internal needle; and
cooling the third material at the distal end of the external needle to −20° C. to −180° C. to freeze the tissue.

21. The method of claim 20, wherein the cryogen is liquid nitrogen.

22. The method of claim 20, wherein the tissue is a nerve.

23. The method of claim 20, wherein the tissue is a tumor.
